(19) United States
(12) Patent Application Publication
(10) Pub. No.: US 2006/0260613 A1
(43) Pub. Date: Nov. 23, 2006

(54) Nasal appliance

(75) Inventor: Mark E. Pinter, Lumberton, NJ (US)

Correspondence Address: SYNNESTVEDT & LECHNER, LLP, 2600 ARAMARK TOWER, 1101 MARKET STREET, PHILADELPHIA, PA, 191072950, US

(73) Assignee: BSA, LLC, Lansdale, PA (US)

(21) Appl. No.: 11/444,662

(22) Filed: Jun. 1, 2006

Related U.S. Application Data
(63) Continuation-in-part of application No. 11/297,136, filed on Dec. 8, 2005.
(60) Provisional application No. 60/634,395, filed on Dec. 8, 2004.

(51) Int. Cl. A61G 10/00; A61M 16/00

(52) U.S. Cl. 128/206.11

(57) ABSTRACT

An appliance adapted to engage and expand nasal passages of a nose and prevent their collapse is disclosed. The appliance includes angularly oriented first and second legs having distal ends insertable into the nasal passageways. A bridge member connects proximal ends of the legs to one another. Third and fourth legs are connected to the first and second legs and have distal ends that engage opposite outer surfaces of the nose. The first and second legs are adjustable in both the coronal and sagittal planes to accommodate noses of various sizes and shapes. Porous substrates and soluble compounds may be attached to the first and second legs and deliver medicaments or other compounds. A method of expanding the nasal passageways by inserting and biasing the appliance into the nose is also disclosed.

### CROSS-REFERENCE TO RELATED APPLICATION

This application is a continuation-in-part of U.S. application Ser. No. 11/297,136, filed Dec. 8, 2005, which claims priority to U.S. Provisional Patent Application No. 60/634,395, filed Dec. 8, 2004. Both the aforementioned parent application and the provisional application are hereby incorporated by reference herein.

### FIELD OF THE INVENTION

The invention concerns a medical appliance insertable into the nasal passages to improve breathing and treat snoring disorders.

### BACKGROUND OF THE INVENTION

The American Academy of Sleep Medicine estimates that 40 to 60 million people suffer from chronic snoring. Snoring is the noise produced during sleep when soft tissue at the back of the nose and throat vibrate. The vibration is induced by the turbulent flow of air through narrowed air passages. Snoring may be an indication of potentially serious medical problems and constitutes a source of annoyance to those in the vicinity of the snorer. The degree to which snoring is considered a problem is evidenced by the number of different surgical procedures, such as somnoplasty, tonsillectomy, and the surgical removal of adenoids or obstructing tissue, developed for its treatment.

Mechanical appliances have been developed as an alternative to surgical treatment of snoring. The nasal passages are, in part, defined by relatively soft cartilaginous tissue, and the interior constitutes a mucous membrane. The relatively soft tissue is prone to collapse under ambient pressure when reduced pressure is present within the nasal passages, as occurs during respiration. The tissue is also sensitive and readily irritated by foreign objects such as appliances inserted to maintain patency of the nasal passages. The presence of the mucous membrane means that the nasal passages can provide a gateway for infection as well as a path for the introduction of medicaments. These factors must be addressed by any device or appliance designed to be inserted into the nasal passages to treat snoring.

### SUMMARY OF THE INVENTION

The invention concerns an appliance adapted to engage and expand the nasal passages of a nose and prevent collapse thereof upon inhalation of air therethrough. The appliance comprises first and second legs arranged in spaced apart relation substantially co-planar with one another. The first and second legs are oriented angularly to one another. Each of the first and second legs have a proximal end, and a distal end insertable within a respective nasal passage. The distal ends are spaced farther apart than the proximal ends for biasing the nasal passages outwardly into an open configuration. A bridge member connects the proximal ends of the first and second legs to one another. Third and fourth legs are arranged in spaced apart relation substantially co-planar with one another. The third and fourth legs each having a proximal end attached to the proximal end of a respective one of the first and second legs, and a distal end engageable with an outer surface of the nose for holding the appliance thereon.

Preferably, the first and second legs each comprise a pair of segments connected to one another by a reverse bend positioned at their respective distal ends. Each segment of each pair is adjustably movable toward and away from one another for establishing a separation distance therebetween, the segments engaging and expanding the nasal passages upon insertion of the device therein. Each of the segment pairs are preferably located in respective sagittal planes relatively to the nose.

The second and third legs each have a loop positioned at their respective distal ends. The loops engage the outer surface of the nose on opposite sides thereof. Preferably, the bridge member comprises a loop positioned between the first and second legs, the loop providing angular adjustability between the first and second legs.

The distal ends of the legs are coated with a polymer coating. Alternately, or in addition to the polymer coating, a porous substrate may be attached to the distal end of at least one of the first and second legs, the substrate adapted to contain a substance for release into the nasal passages. The substrate may comprise, for example, an aromatic compound or a medicament. In another embodiment, a soluble membrane may be attached to the distal end of at least one of the first and second legs. The soluble membrane is adapted to contain a substance for release into the nasal passages.

The invention also encompasses a method of expanding the nasal passages of a nose into an open configuration. The method comprises:

(a) inserting a distal end of a first and a second leg into respective nasal passages;

(b) biasing the distal ends of the first and second legs outwardly away from one another, each leg thereby engaging a sidewall of the respective nasal passages;

(c) engaging a distal end of a third and a fourth leg with respective outer surfaces of the nose on opposite sides thereof; and

(d) biasing the distal ends of the third and fourth legs inwardly against the respective outer surfaces.

The method may also include:

(a) forming the first and second legs from respective pairs of segments;

(b) orienting the segments of each pair in respective sagittal planes relatively to the nose; and

(c) biasing the segments away from one another in the respective sagittal planes, the segments engaging and expanding the nasal passages in the respective sagittal planes.

### BRIEF DESCRIPTION OF THE DRAWINGS

FIG. 1 is a top view of an embodiment of a nasal appliance according to the invention;

FIG. 2 is a side view of the nasal appliance shown in FIG. 1;

FIG. 3 is a cross sectional view of the nasal appliance taken at line 3-3 of FIG. 1;

FIG. 4 illustrates the nasal appliance shown in FIG. 1 in use; and

FIGS. 5 and 6 show top views of alternate embodiments of the nasal appliance according to the invention.

### DETAILED DESCRIPTION OF THE EMBODIMENTS

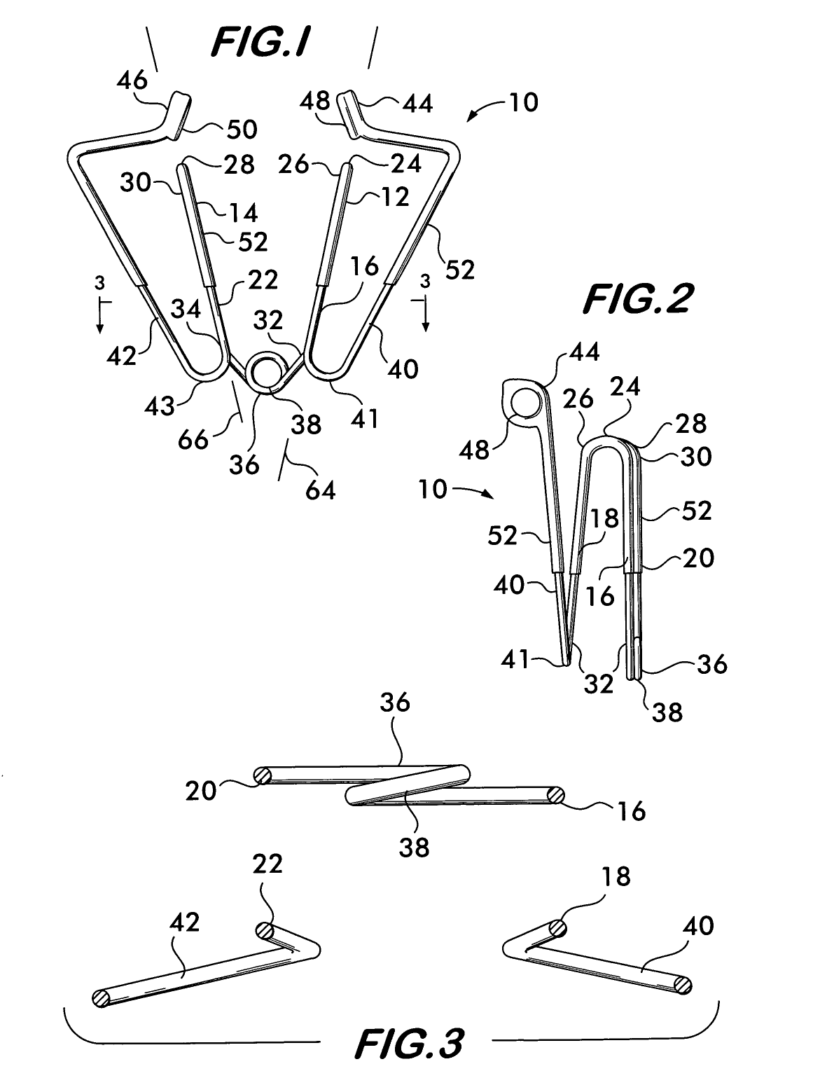

FIG. 1 illustrates a nasal appliance 10 according to the invention. Nasal appliance 10 comprises first and second legs 12 and 14 arranged in spaced apart relation. The first and second legs are substantially co-planar as shown in FIG. 2, and are preferably formed by pairs of segments 16 and 18 and 20 and 22 (see FIG. 3). The segments 16 and 18 comprising leg 12 are joined to one another by a reverse bend 24 at the distal end 26 of leg 12. The segments 20 and 22 are joined to one another by another reverse bend 28, also at the distal end 30 of the leg 14. As shown in FIGS. 1 and 2, the legs 12 and 14 have respective proximal ends 32 and 34. Preferably, the distal ends 26 and 30 of the first and second legs 12 and 14 are spaced wider apart than the proximate ends of these legs, providing an angular orientation between the first and second legs.

The proximal ends of legs 12 and 14 are connected to one another by a bridge element 36. As best shown in FIG. 1, the bridge element 36 comprises a loop 38 that provides flexibility and resilience to the legs 12 and 14, allowing their relative angular position to be adjusted to accommodate a nasal passage as described below.

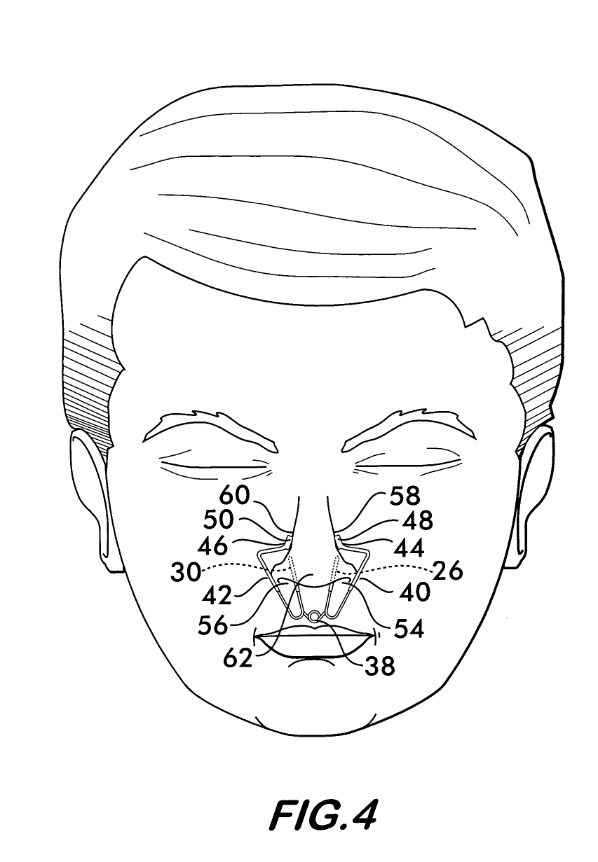

Third and fourth legs 40 and 42 are attached, respectively, to the proximal ends 32 and 34 of the first and second legs 12 and 14. The third and fourth legs are also arranged angularly with respect to one another and are substantially co-planar, as shown in FIG. 2. The plane of the third and fourth legs is preferably displaced from the plane of the first and second legs, thereby providing a geometry that best fits a nose as described in detail below. The respective distal ends 44 and 46 of the third and fourth legs 40 and 42 project inwardly so as to engage opposite sides of the nose as illustrated in FIG. 4. Preferably, loops 48 and 50 (see especially FIGS. 1 and 2) are positioned respectively at the distal ends of the third and fourth legs 40 and 42. The loops enlarge the engagement area between the third and fourth legs and the outside of the nose, thereby mitigating the potential for irritation. The loops may have any shape, for example, round as shown, oval or polygonal.

Appliance 10 is preferably formed integrally from FDA approved surgical grade stainless steel wire having a diameter between about 0.030 and 0.036 inches. Stainless steel is preferred because it is radiopaque, will not corrode and is compatible with living tissue. Surgical grade wire, as used in orthodontic work, provides a material with great flexibility and resilience, allowing the appliance to be repeatedly bent and adjusted in shape for comfort of the user and effectiveness in supporting and expanding the nasal passageways. The aforementioned diameter range has been found to provide excellent fatigue life and adequate stiffness to support and expand the cartilaginous tissue of the nose.

For comfort and safety, the appliance 10 is formed with rounded bends of generous radii; sharp points or edges that might irritate or cut nasal tissue, especially the sensitive internal lining of the nasal passageways, are avoided. Further protection to the user is provided by coating at least those portions of the appliance that contact the nose with a polymer coating 52. The coating 52 is positioned on the distal ends of legs 12, 14, 44 and 46 and preferably comprises polyvinylchloride resin. Polyvinylchloride is preferred because it is flexible, hypo-allergenic, color fast, chemically inert, and able to withstand high temperatures thereby allowing the device to be sterilized in an autoclave. This polymer also provides a smooth surface with few irregularities where bacteria or viruses can accumulate. These characteristics provide a sanitary appliance that will not irritate or infect the mucous membrane of the nasal passageways when used properly. The coating 52 may be applied by dipping the legs into a molten bath of the polymer and then allowing it to cure. Coating thicknesses of not less than 0.030 inches per side (0.060 inches total) are preferred. Other polymers are also feasible, for example, resins such as methylmethacrylate.

In use, as illustrated in FIG. 4, the distal ends 26 and 30 of the first and second legs 12 and 14 are inserted into the nasal passageways 54 and 56, and the distal ends 44 and 46 of the third and fourth legs 40 and 42 are engaged with the outer surfaces 58 and 60 on opposite sides of nose 62. The appliance 10 displays a distinct “W” shape in plan view. This shape is advantageous because it supports and expands the nasal passages 54 and 56 outwardly without impinging on and irritating the nasal septum. The resilience and flexibility of the surgical steel coupled with the preferred diameter of the wire forming the appliance allows the device to be bent and adjusted so as to control the angular orientation of the legs 12 and 14 to bias the nasal passageways outwardly sufficiently to prevent snoring, yet comfortably. The loop 38 of bridge element 36 allows for excellent adjustability and resilience of the legs 12 and 14, and acts as a spring to bias the legs outwardly against the cartilaginous tissue. The third and fourth legs 40 and 42 are in a different plane from the first and second legs (see FIG. 2) so as to adapt to the geometry of the nose 62. These legs are also angularly oriented, and biased inwardly so that loops 48 and 50 engage the nose surfaces 58 and 60 firmly yet comfortably to reliably retain the appliance in the nose and prevent it from being aspirated accidentally. Reverse bends 41 and 43 are used to attach the legs 40 and 42 respectively to the legs 12 and 14. The reverse bends 41 and 43 permit angular adjustment of the third and fourth legs 40 and 42 independently of the first and second legs to ensure proper engagement of the loops 48 and 50 with the nose. The legs 40 and 42 are also of a size and orientation such as to make accidental ingesting or swallowing of the appliance very difficult. In the unlikely event that this happens, the radiopaque nature of the device will allow easy detection by X-ray techniques.

While the “W” shape and bridge loop 38 allow for substantial lateral adjustment of the appliance (toward and away from the nasal septum), adjustment in the fore and aft directions (parallel to the nasal septum) is also provided by constructing legs 12 and 14 from segment pairs 16 and 18 and 20 and 22 as best shown in FIG. 2. As noted above, each segment pair is located in a respective sagittal plane 64 and 66 (see also FIG. 1). The reverse bends 24 and 28 of the legs allow the segments to be separated or narrowed in their respective sagittal planes to expand and support the nasal passageways in the direction parallel to the nasal septum as required for comfort and breathing efficiency. Thus, the appliance provides the ability to expand the nasal passageways in two mutually perpendicular directions to ensure proper breathing and prevent snoring. The infinite adjustability of the appliance, the curved ends and loops of the legs, and the absence of sharp points and edges allows it to be adapted comfortably and safely to any nose size.

In addition to treatment of snoring, the appliance 10 according to the invention may be used, for example, to aid in breathing when the nose has suffered trauma and is healing, such as post surgery or after an injury. Other forms of treatment can also be realized with minor modifications to the appliance.

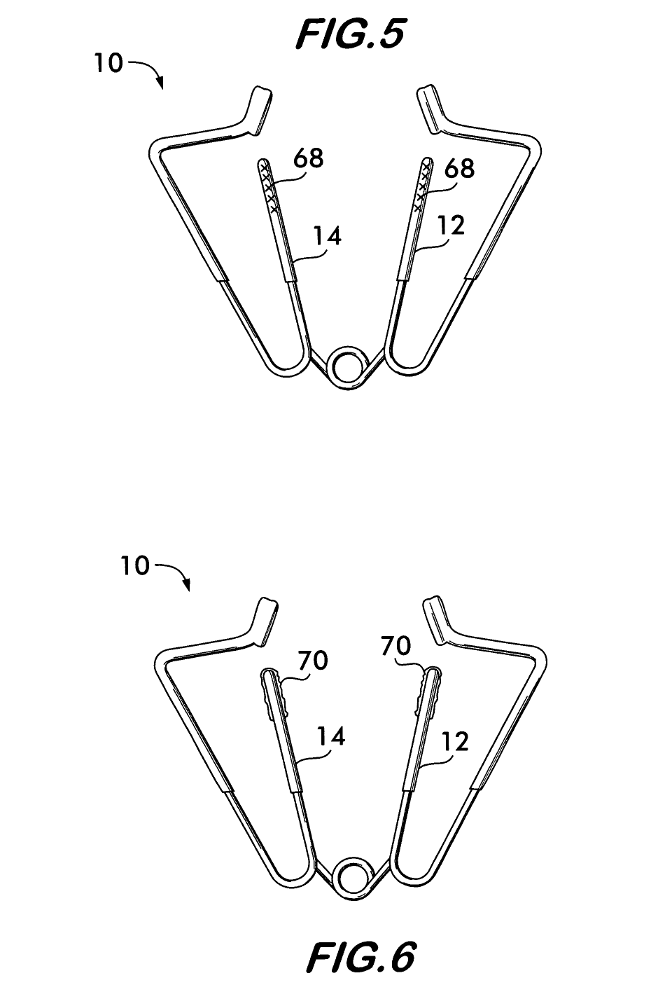

As shown in FIG. 5, a porous substrate 68 may be attached to one or both of the legs 12 and 14. The substrate may be impregnated with a substance, such as an aromatic compound or a medicament. When an aromatic compound is used, the appliance 10 is an effective delivery device for aroma therapy. Aromatic compounds smelling like chocolate, coffee and other pleasant fragrances are feasible. Medicaments such as antihistamines, vitamin B12 for the treatment of anemia, calcium for the treatment of osteoporosis, anti-asthma compounds, medications for treatment of diabetes and mentholated ointments may be delivered by the substrates 68 of the appliance. In another embodiment, shown in FIG. 6, the legs 12 and 14 may be coated with soluble membrane 70, such as ethyl acetate cellulose. The membrane may be the vehicle for a medicament, which is absorbed into the bloodstream through the mucous membrane of the nose as the membrane dissolves when the appliance is inserted into the nasal passageways.

Nasal appliances according to the invention provide a safe, comfortable and effective device for treatment of snoring as well as other disorders.

What is claimed is:

1. An appliance adapted to engage and expand nasal passages of a nose and prevent collapse thereof upon inhalation of air therethrough, said appliance comprising:
first and second legs arranged in spaced apart relation substantially co-planar with one another, said first and second legs being oriented angularly to one another, each of said first and second legs having a proximal end, and a distal end insertable within a respective nasal passage, said distal ends being spaced farther apart than said proximal ends for biasing said nasal passages outwardly into an open configuration;
a bridge member connecting said proximal ends of said first and second legs to one another; and
third and fourth legs arranged in spaced apart relation substantially co-planar with one another, said third and fourth legs each having a proximal end attached to said proximal end of a respective one of said first and second legs, and a distal end engageable with an outer surface of said nose for holding said appliance thereon.

2. An appliance according to claim 1, wherein said third and fourth legs are oriented angularly with respect to one another.

3. An appliance according to claim 1, wherein said first and second legs occupy a first plane and said third and fourth legs occupy a second plane offset from said first plane.

4. An appliance according to claim 1, wherein said first and second legs each comprise a pair of segments connected to one another by a reverse bend positioned at their respective distal ends, each segment of each pair being adjustably movable toward and away from each other for establishing a separation distance therebetween, said segments engaging and expanding said nasal passages upon insertion of said device therein.

5. An appliance according to claim 4, wherein each of said segment pairs are located in respective sagittal planes relatively to said nose.

6. An appliance according to claim 1, wherein said second and third legs each have a loop positioned at their respective distal ends, said loops engaging said outer surface of said nose on opposite sides thereof.

7. An appliance according to claim 6, wherein said loops have a substantially circular shape.

8. An appliance according to claim 1, wherein said bridge member comprises a loop positioned between said first and second legs, said loop providing angular adjustability between said first and second legs.

9. An appliance according to claim 8, wherein said legs and said bridge member are integrally formed from a unitary piece of wire.

10. An appliance according to claim 1, wherein said distal ends of said legs are coated with a polymer coating.

11. An appliance according to claim 1, further comprising a porous substrate attached to said distal end of at least one of said first and second legs, said substrate adapted to contain a substance for release into said nasal passages.

12. An appliance according to claim 11, wherein said substance comprises an aromatic compound.

13. An appliance according to claim 11, wherein said substance comprises a medicament.

14. An appliance according to claim 1, further comprising a soluble membrane attached to said distal end of at least one of said first and second legs, said soluble membrane adapted to contain a substance for release into said nasal passages.

15. An appliance according to claim 1, wherein said soluble membrane comprises ethyl acetate cellulose.

16. An appliance adapted to engage the nasal passages of a nose, said appliance comprising:
a first pair of legs, each having proximal ends attached to one another, and distal ends in spaced relation to one another, said distal ends being respectively insertable within said nasal passages for biasing them outwardly into an open configuration; and
a second pair of legs, each having a proximal end attached to a proximal end of one of said first pair of legs, each leg of said second pair having a distal end projecting inwardly toward said distal ends of said first pair of legs, said distal ends of said second pair being engageable with said nose on opposite sides thereof for holding said appliance thereon.

17. An appliance according to claim 16, wherein said legs comprising said first pair are angularly oriented with respect to one another such that said distal ends are further apart than said proximal ends.

18. An appliance according to claim 16, wherein said distal ends of said second pair of legs each comprise a loop engageable with said nose.

19. An appliance according to claim 16, wherein said proximal ends of said first pair of legs are attached to one another by a flexible loop, said loop permitting angular adjustability of said first pair of legs relatively to one another.

20. An appliance according to claim 16, wherein said first pair of legs define a first plane and said second pair of legs define a second plane, said first and second planes being offset from one another.

21. An appliance according to claim 16, wherein a portion of said leg pairs are coated with a polymer coating.

22. An appliance according to claim 16, wherein each leg of said first pair comprises a pair of segments connected to one another by a reverse bend positioned at their respective distal ends, said segments being adjustably movable toward and away from one another and engageable with said nasal passages for expanding them into said open configuration.

23. An appliance according to claim 22, wherein each said pair of segments is located in a respective sagittal plane relatively to said nose, adjustably moving said segments expanding each said nasal passages in one of said sagittal planes.

24. An appliance according to claim 16, wherein said appliance is formed from a continuous wire.

25. An appliance according to claim 24, wherein said wire comprises surgical grade stainless steel.

26. A method of expanding the nasal passages of a nose in an open configuration, said method comprising:
inserting a distal end of a first and a second leg into respective nasal passages;
biasing said distal ends of said first and second legs outwardly away from one another, each said leg thereby engaging a sidewall of said respective nasal passages;
engaging a distal end of a third and a fourth leg with respective outer surfaces of said nose on opposite sides thereof; and
biasing said distal ends of said third and fourth legs inwardly against said respective outer surfaces.

27. A method according to claim 26, further comprising:
forming said first and second legs from respective pairs of segments;
orienting said segments of each pair in respective sagittal planes relatively to said nose; and
biasing said segments away from one another in said respective sagittal planes, said segments engaging and expanding said nasal passages in said respective sagittal planes.
